(19) United States
(12) Patent Application Publication
(10) Pub. No.: US 2004/0241698 A1
(43) Pub. Date: Dec. 2, 2004

(54) Method for producing a normalized gene library from nucleic acid extracts of soil samples and the use thereof

(76) Inventors: Berhard Hauer, Fussgonheim (DE); Markus Matuschek, Weinheim (DE); Rolf Schmid, Stuttgart (DE); Christiane Buta, Stuttgart (DE); Isabelle Kauffmann, Stuttgart (DE); Katrin Lammle, Vaihingen (DE); Hubert Zipper, Stuttgart (DE)

Correspondence Address: LAHIVE & COCKFIELD, LLP., 28 STATE STREET, BOSTON, MA, 02109, US

(21) Appl. No.: 10/490,304

(22) PCT Filed: Sep. 19, 2002
(86) PCT No.: PCT/EP02/10510

(30) Foreign Application Priority Data
Sep. 21, 2001 (DE) 10146572.6

(51) Int. Cl. C12Q 1/68; C07H 21/04

(52) U.S. Cl. 435/6; 536/23.7

(57) ABSTRACT

The present invention relates to a method for preparing a normalized gene library from nucleic acid extracts of soil samples and to gene structures and vectors used in said method. The invention further relates to the use of the normalized gene library for the screening of genes coding for novel biocatalysts from the soil samples.

The present invention relates to a method for preparing a normalized gene library from nucleic acid extracts of soil samples and to the use thereof.

Enzymes derived from microorganisms have potential for broad application. In the medical-pharmaceutical sector, enzymes are used, for example, in drug screening research and in the development of molecular biological assay systems. Enzymes are used in synthesis of antibiotics and derivatives thereof, for preparing hormones and as additives in the food industry, in the detergent industry and as catalysts for producing chemicals, to name but a few examples. In order to improve the current enzymic methods and to develop new fields of application for enzymes, it is necessary to optimize present enzymes and to select novel enzymes through screening.

Previously, screening for novel enzymes has been limited by the fact that only pure cultures of microorganisms were screened. However, it was shown that only approx. 1% of all microorganisms can be cultured, and 99% of microorganisms cannot be cultured as pure strains by using the currently known methods. Consequently, the latter organisms have previously not been available for isolation of novel enzymes. Gene libraries of nucleic acids of various environmental locations theoretically comprise any enzymes occurring in said location, without the need for the donor organisms in question to be isolated.

A method for preparing gene libraries from environmental samples must meet specific demands:

the method must be capable of isolating DNA from all species present in the sample.

to generate the gene library, the DNA must be intact after isolation and must not be damaged by the various purification and isolation processes.

the method must be independent of the composition of the soil sample and of the population of microorganisms.

It is critical here, for example, to establish a balance between, on the one hand, comprehensive cell lysis and, on the other hand, as little destruction of the DNA by shear forces as possible. Examples of isolating DNA from soil samples are described in Moré et al. (Appl. Environm. Microbiol., May 1994, 1572-1580) and Zhou et al. (Appl. Environm. Microbiol., Feb. 1996, 316-322). However, here either the nucleic acids are extracted from the organic material in their entirety, i.e. nonselectively, or merely DNA of Gram-positive organisms is isolated.

The isolated DNA must be clonable. One problem when isolating DNA from soil is, for example, that nucleic acid preparations contain an increased amount of humic substances which greatly impair or even render impossible further treatment of the nucleic acids, for example quantification or further enzymic treatment.

Furthermore, it is essential to ensure that those nucleic acid species in the isolated nucleic acid population, which are by nature less commonly present, are not lost during further work-up such as, for example, cloning into suitable vectors for generating a gene library. This can be achieved by preparing a normalized gene library, during the generation of which the concentration of frequently occurring DNA species is reduced and that of rarely occurring DNA species is increased. Numerous methods for increasing the concentration of rarely occurring DNA species are known from the literature. WO 95/08647, WO 95/11986, WO 97/48717 and WO 99/45154 are mentioned by way of example.

WO 95/08647 first discloses preparation of a cDNA gene library in a suitable vector and provision of the plasmids in their single-stranded form by denaturation. This is followed by preparing fragments which are complementary to noncoding 3′ regions of the single-stranded plasmids and by hybridization thereof with the cDNA gene library. Selection here is based on the principle that, statistically, the noncoding 3′ regions occur less frequently in the genome than coding DNA regions which are often conserved. The hybrids formed are purified and subjected to further denaturation and reassociation cycles. However, the previously described procedure demands detailed knowledge with respect to the noncoding nucleotide sequences. WO 95/08647 aims at providing a normalized human cDNA catalog, starting from mammalian cells, in particular from cells of the brain, the lung or the heart. The isolation of microbial genomic DNA from soil samples is not mentioned; rather, the starting material of WO 95/08647 is isolated mRNA.

WO 95/11986 discloses a method for preparing a subtractive cDNA gene library, which likewise comprises cloning in a first step total DNA in the form of cDNA into a vector. Subsequently, said DNA is denatured and the single-stranded cDNA is used for hybridization with the specifically labeled nucleic acid molecule which is to be subtracted from the total DNA. Removal of the labeled DNA hybrids formed produces a subtractive DNA library. However, this does not increase the concentration of less commonly occurring nucleic acid species in the remaining DNA library. Moreover, the DNA used here is isolated from mammalian cells, in particular tumor cells, the starting material used being isolated mRNA. The isolation of microbial genomic DNA and normalization thereof are not mentioned.

In contrast to the previously discussed methods for preparing subtractive gene libraries by means of hybridization with probes or nucleic acid fragments prepared for that purpose, WO 97/48717 discloses the preparation of a normalized DNA gene library, in which the starting material used is genomic DNA of nonculturable organisms, for example from soil samples. Here, the DNA is isolated by means of proteinase K and “freeze-thaw” methods, then purified via a CsCl gradient and concentrated via PCR, followed by studying the complexity of the gene library by way of 16S-rRNA analysis and, finally, normalizing said gene library by way of denaturation and reassociation at 68° C. for 12-36 hours. However, this US document lacks information about when the optimal moment for stopping reassociation actually occurs, despite this being crucial for the optimal yield of less commonly present DNA species. The latter likewise applies to the document WO 99/45154.

Another problem when preparing a normalized gene library is the fact that the availability of suitable recognition sites for restriction endonucleases for the purpose of cloning the DNA fragments into a suitable vector is greatly limited. The reason for this is primarily the intention of not fragmenting the isolated DNA fragments encompassing a particular size range again in the course of preparation for cloning. For this reason, the isolated DNA fragments are subjected in conventional methods to enzymic methylation which is intended to protect the DNA against attack by restriction endonucleases. A problem, however, is that said methylation is very complicated and, moreover, there is no 100% guarantee of a uniform distribution thereof over the entire DNA, so that in practice the protection against attack by restriction endonucleases is only unsatisfactory (Robbins, P. W. et al. (1992) Gene 111: 69-76).

It is an object of the present invention to provide a method for preparing gene libraries and to provide gene constructs both of nonculturable and culturable organisms. In particular, the method of the invention is intended to provide the possibility of preparing gene libraries from soil samples in order to also provide rarely occurring DNA of organisms which previously were not capable of being cultured in the laboratory. Another object of the present invention is the identification of novel biocatalysts from soil samples.

We have found that this object is achieved by a method for preparing a normalized gene library from nucleic acid extracts of soil samples, which method comprises

a) extracting nucleic acids from living organisms present in soil samples,

b) fragmenting said nucleic acids,

c) quantifying the nucleic acid fragments by means of fluorescent dyes,

d) normalizing said nucleic acid fragments, first denaturing the latter and then monitoring the course of renaturation by means of fluorescent dyes,

g) separating, after renaturation has ended, the double-stranded nucleic acids from the single-stranded nucleic acids by adsorption chromatography, the amount of nucleic acid species present in the fraction of the single-stranded nucleic acids being frequently approximately equal (normalized),

e) generating the gene library by cloning the normalized nucleic acid species into a vector.

One advantage of the present invention is the fact that the nucleic acids are extracted from the soil samples, fragmented, quantified, normalized and then cloned into a vector suitable for cloning, amplification and/or expression. As illustrated in more detail hereinbelow, methylation of the isolated DNA for protection against unwanted fragmentation by restriction endonucleases is not required according to the invention, since fragmentation takes place before normalization. Advantageously according to the invention, special “linkers” which possess recognition sequences for extremely rarely cleaving restriction endonucleases are attached to the restriction fragments after fragmentation. This considerably simplifies the method of the invention with a simultaneous increase in the efficiency of gene library preparation.

Another advantage of the method of the invention here is the fact that nucleic acids are extracted from soil-dwelling organisms which have not previously been cultured in the laboratory. Examples of previously nonculturable known microorganisms to be mentioned are: bacteria in the rumen of ruminants, obligate endosymbionts of protozoa and insects, the magnetotactic bacterium Achromatium oxaliferum (Amann, R. I. et al. (1995) Microbiol. Rev. 59: 143-169).

The method of the invention is distinguished by selective isolation of nucleic acids from actinomycetes. The specific knowledge or at least the specific exclusion of groups of organisms from soil samples is advantageous in that a suitable host organism into which the isolated DNA fragments are, where appropriate, to be transferred later (e.g. for the purpose of cloning or functionality control by expression) can be optimally selected.

In an advantageous variant of the present invention, DNA is first isolated according to a protocol by Zhou et al. (1996, Appl. Environ. Microbiol., 62(2): 316-322), modified according to the invention, said modifications comprising carrying out the freeze-thaw cycles prior to proteinase K treatment. This type of sample treatment makes it possible to virtually rule out isolation of DNA from actinomycetes, i.e. the DNA isolated in this manner is advantageously suitable for transfer into Gram-negative microorganisms such as E. coli, for example.

The inventive method for preparing a normalized gene bank from soil samples is particularly advantageous in that it is possible to control DNA isolation so as for the latter to be selective with respect to the groups of organisms occurring in soil samples. Thus, according to the invention, it is possible, for example, to selectively isolate DNA from actinomycetes by sequential DNA isolation, i.e. firstly according to Zhou et al. and then according to Moré et al. (1994, Appl. Environ. Microbiol., 60(5): 1572-1580). To this end, the cells in a manner are first disrupted according to Zhou et al., modified according to the invention, resulting in the DNA being extracted from the microorganisms with the exception of the actinomycetes. An incubation with SDS is followed by a centrifugation step. The actinomycete cells which have not yet been disrupted are now in the pellet. After a washing step, the DNA is extracted from this cell pellet by the method according to a protocol of Moré, which has been modified according to the invention. This involves, for example, using a mixture of glass beads 0.1-0.25 mm in diameter and purifying the DNA by means of silica, rather than carrying out ethanol precipitation. This procedure according to the invention is particularly advantageous when the DNA is subsequently to be cloned into streptomycetes, Rhodococcus or Corynebacterium.

It is thus an advantage of the method of the invention that there are separate fractions, namely the supernatant and pellet of the abovementioned centrifugation step, from which DNA can be isolated which does (pellet) or does precisely not (supernatant) originate predominantly from actinomycetes.

An advantageous variant of the present invention involves fragmenting the nucleic acids extracted from the soil samples into fragments of a size range of about 1-10 kb, preferably of about 2-9 kb, and particularly preferably of about 3-8 kb. This is carried out according to common methods, for example in a partial restriction mixture with the endonucleases Sau3AI or Hsp92II and subsequent size fractionation via gel electrophoresis.

In a variant of the present method, the nucleic acids are fragmented with the addition of nonacetylated bovine serum albumin (BSA). Depending on the composition of the soil sample used for disruption, it may be that not all of the contaminants, inter alia humic substances, are sufficiently removed from the nucleic acid solution during the purification procedure. The addition of nonacetylated BSA minimizes inhibition of the restriction endonucleases by humic substances present in the nucleic acid extract. A final concentration of nonacetylated BSA of about 1-15 μg, preferably of about 2-12 μg, and particularly preferably of about 10 μg, per μl of restriction mixture is advantageous here. The amount of nonacetylated BSA to be used may furthermore be tested separately, depending on the restriction enzyme (and production batch, where appropriate) used, and may, in the individual case, also deviate from the abovementioned values.

The inventive method for preparing a normalized gene library is further distinguished by using in step c) fluorescent dyes, preferably SYBR-Green-I, for quantifying the nucleic acids extracted from soil samples and/or their fragments. This is a particular advantage of the method of the invention, since the aqueous crude extract of a digested soil sample has, inter alia, a high humic substances content which makes photometric quantification of the DNA in said crude extract impossible, since the humic substances also strongly absorb in the UV region, for example at 260 nm. The method of the invention solves this problem by quantifying the DNA with the aid of fluorescent dyes, preferably SYBR-Green-I (Molecular Probes, Inc. USA). It is generally possible for the results in determinations by means of fluorescence spectroscopy to be distorted due to contamination, in this case, for example, humic substances, which cause fluorescence quenching. It is an advantage of the present invention that said quenching can be eliminated by diluting the crude extract by an order of magnitude of about 1:30 to 1:50 and by common standard addition methods (Skoog, D. A., Leary, J. J.: Instrumentelle Analytik—Grundlagen, Geräte, Anwendungen; pp. 176f, 1^{st} edition, Springer-Verlag, Berlin Heidelberg N.Y.). Thus, according to the invention, the remaining error in the determination of DNA from soil samples is only about 10%.

In a particularly advantageous variant of the present invention the fragmented nucleic acids are linked to linkers which have at least one recognition site for a rarely occurring restriction endonuclease. According to the invention, the linkers are ligated to the fragmented nucleic acids, before the DNA is normalized, i.e. prior to step d) of the abovementioned method. Preferably, the linkers, and preferably also the vector used, for example, for cloning and/or amplifying the isolated DNA, have a gene structure which in turn has a recognition site for the restriction endonuclease I-Ppol. The I-Ppol endonuclease requires a recognition sequence of at least 15 base pairs (bp) in length. This ensures that the enzyme cleaves the nucleic acid used for restriction only extremely rarely, if at all. Thus, the genome of the E. coli bacterium does not contain any recognition site for I-Ppol, and Saccharomyces cerevisiae “cleaves” only three times in the genome of the yeast I-Ppol. However, other rarely occurring recognition sites for restriction endonucleases are also conceivable in principle according to the invention. That is to say that, alternatively, other endonucleases having extremely long recognition sequences could also be used according to the invention, such as “homing endonucleases”, for example. It is further also conceivable according to the invention that the recognition sites of a restriction endonuclease in the linkers and in the vector are, although not identical, at least compatible. The present invention therefore also relates to a method which is distinguished by using in step f) a vector which has at least one recognition site for a rarely occurring restriction endonuclease, which is compatible with the recognition site in the linkers. SEQ ID No. 2 and 3 depict by way of example linkers preferred according to the invention.

The present invention therefore also relates to a gene structure comprising at least one multiple cloning site with at least one rarely occurring recognition site for restriction endonucleases, a primer-binding site and/or a T7-polymerase recognition site whose activity is regulated via the lac operator and which can be used for increased expression of the cloned soil DNA. A variant of the gene structure, which is advantageous according to the invention, comprises at least one recognition site for the I-Ppol restriction endonuclease. In a preferred variant of the present invention, the gene structure of the invention is distinguished by having a sequence according to SEQ ID No. 1.

The present invention further relates to the use of the rarely occurring recognition site for the I-Ppol restriction endonuclease for preparing a gene structure of the invention.

The present invention therefore also relates to a vector which has at least the previously characterized gene structure and also additional nucleotide sequences for selection, for replication in the host cell and/or for integration into the host cell genome. The literature describes numerous examples of suitable vectors such as, for example, plasmids of Bluescript series, e.g. pBluescript SK+ (Short, J. M. et al. (1988) Nucleic Acids Res. 16: 7583-7600; Alting-Mees, M. A. and Short, J. M. (1989) Nucleic Acids Res. 17: 9494), pJOE930 (Altenbuchner J. et al. (1982) Meth. Enzymol. 216: 457-466), pUC18 or 19 (Vieira, J. & Messing, J. (1982) Gene 19:259; Yanisch-Perron, C. et al. (1985) Gene 33: 103).

A normalized gene library is generated by increasing the concentration of the naturally rarer DNA species and, accordingly, reducing the concentration of the frequently occurring DNA species. This is carried out in principle by denaturation of the dsDNA isolated from the soil samples and subsequent renaturation over a certain period, with the frequently occurring DNA species rehybridizing faster than the rare ones. When normalizing DNA, judging the moment at which to stop renaturation is critical in order to achieve an optimal ratio between rarely occurring and frequently occurring DNA species so that theoretically all DNA species are present in the same amount. If this step of ssDNA/dsDNA separation is carried out too early, the efficiency of normalization is only low, since a large proportion of ssDNA still consists of frequently occurring DNA molecules of the same kind and of one type of organism. If, on the other hand, ssDNA/dsDNA separation is carried out too late, the entire ssDNA may already have rehybridized and is present in the double-stranded form. The result of this is the serious disadvantage that it is not possible to isolate a sufficient amount of ssDNA for further treatment and that, moreover, the complete range of the rare DNA molecules actually occurring in the soil sample is not represented.

It is therefore a particular advantage of the method of the invention to be able to monitor the time course of renaturation of the previously denatured nucleic acid fragments.

According to the invention, this is carried out fluorometrically with the aid of DNA-specific fluorescent dyes. Preference is given here according to the invention to SYBR-Green-I. SYBR-Green-I has the advantage of distinguishing qualitatively between ssDNA and dsDNA. This is possible owing to the different fluorescence yields of these two DNA species when complexed with the dye. The [dsDNA-SYBR-Green-I] complex has a significantly higher, sometimes up to 13 times higher, fluorescence than the corresponding ssDNA-dye complex.

According to the invention, aliquots are removed from the “normalization mixture” during rehybridization, admixed with SYBR-Green-I, and the fluorescence is compared to the fluorescence of the nondenatured control mixture having the same DNA concentration. In the course of renaturation, the relative fluorescence increases owing to the increasing dsDNA content. When rehybridization of ssDNA to dsDNA is complete, the original fluorescence level of the nondenatured sample is reached. A problem with the above-described procedure is the sampling and, respectively, the impairment of the hybridization conditions, which may occur in the process, and the composition of the rehybridization buffer. The present invention solves this problem in an advantageous manner, an only very small sample volume of about 1-5 μl, preferably 1.5-3 μl, particularly preferably of 1.8-2.5, and most particularly preferably of 2 μl, being sufficient for fluorescence spectroscopy. In addition, the pipette tips are preheated to a temperature which corresponds to the hybridization temperature, in order to prevent inaccurate sampling and a decrease in hybridization temperature. One variant of the invention uses a hybridization buffer comprising no more than 0.01%, preferably from 0.0001 to 0.01%, particularly preferably from 0.0001 to 0.001%, SDS (v/v) and a sodium chloride concentration of between 0.1 M and 1.5 M, preferably from 0.2 M to 1.0 M, particularly preferably from 0.3 M to 0.8 M, and in particular of 0.4 M.

In one variant of the present invention, it is thus possible, owing to the procedure illustrated and, for example, based on a concentration used of 1 μg/μl of size-fractionated E. coli DNA (3-6 kb), to determine an optimal moment for stopping rehybridization in the range of about 70-220 minutes, preferably of about 80-200 minutes and particularly preferably of about 100-140 minutes.

After denaturation and subsequent renaturation (rehybridization) have ended, the DNA still present in single-stranded form (ssDNA) is removed from the renatured double-stranded DNA (dsDNA) and amplified by means of PCR, for example. Repeating the above-described inventive steps of normalization several times results in the desired increase in concentration of rarely occurring DNA species from soil samples with simultaneous decrease in concentration of the more common DNA species so that fractions of nucleic acid species are obtained, in which all DNA species are frequently present in approximately equal (normalized) amounts.

There are in principle various possibilities for removing ssDNA from dsDNA available, such as, for example, adsorption chromatography (e.g. by means of silica gel or hydroxyapatite) or dsDNA fragmentation by means of restriction endonucleases.

Preference is given according to the invention to a method for preparing a normalized gene library from soil samples, in which method adsorption chromatography is carried out by means of hydroxyapatite (crystalline calcium phosphate [Ca₅(PO₄)₃OH]₂). In an advantageous variant of the inventive method for preparing a normalized gene library from soil samples, adsorption chromatography is carried out in a batch process rather than in the usual column form.

In another variant of the present invention, adsorption chromatography is carried out in (spin) columns (e.g. empty Mobicol columns from MoBiTec, Göttingen, Germany) which are packed with hydroxy apatite suspension.

In the batch process, the ssDNA is removed according to the invention by adding from 10 to 100 μl of hydroxy apatite suspension, preferably 25-80 μl, and particularly preferably 40-60 μl, per 1 μg of DNA. Examples of possible containers in which removal in the batch process can take place are PCR reaction vessels (0.2 ml) or standard reaction vessels (1.5 or 2 ml).

In order to achieve that only dsDNA binds to the hydroxyapatite and ssDNA remains in the supernatant, the entire DNA mixture (rehybridization mixture) is taken up according to the invention in ssDNA elution buffer (medium salt buffer, e.g. 0.15-0.17 M NaPO₄; pH 6.8) at room temperature and applied to the hydroxyapatite which has likewise been suspended in ssDNA elution buffer. ssDNA and dsDNA are fractionated according to the invention at temperatures of from 20° C. to 60° C., preferably from 20° C. to 30° C., and particularly preferably of 22° C. (RT).

To remove ssDNA at RT, the DNA mixture is taken up in ssDNA elution buffer, 0.17 M NaPO₄ (pH 6.8), at RT. For a removal at 60° C., it is taken up in 0.15 M ssDNA elution buffer. Elution of the bound dsDNA is carried out using 0.34 M NaPO₄ (pH 6.8) (dsDNA elution buffer). After applying the buffer and short centrifugation, the desired DNA in each case is present in the supernatant.

In a variant of this method, the removal is carried out on spin columns at room temperature. Here, the rehybridization mixture taken up in ssDNA elution buffer is applied to spin columns packed with 50-100 μl of hydroxyapatite suspension (suspended in ssDNA elution buffer). After centrifugation, the ssDNA is present in the eluate; after applying dsDNA elution buffer, the bound dsDNA may likewise be eluted by centrifugation.

The previously illustrated procedure of the invention in the batch process is distinguished from conventional column chromatography by the advantages that it is possible to process a larger number of samples, that the fractionation is more constantly and easily temperature-controllable and is also faster. Furthermore, it is overall easier to manage than the methods described in textbooks, for example by Maniatis (Maniatis V., Sambrook J, Fritsch E F & Maniatis V (1989). Molecular Cloning: A Laboratory Manual. Vol. I-III. Cold Spring Harbour Laboratory Press).

As illustrated above, the desired rarely occurring ssDNA is present in the supernatant or eluate after centrifugation of the hydroxyapatite mixture and, where appropriate after purification via common methods of gel chromatography (e.g. Sephadex) or butanol extraction, may be used for further processing such as, for example, PCR or cloning into a suitable vector, resulting in a normalized gene library of nucleic acids of soil-dwelling microorganisms.

The present invention further relates to the use of the normalized gene library prepared according to the invention for identifying genes coding for novel biocatalysts from soil-dwelling microorganisms. Appropriate procedures for screening a gene library for identifying novel biocatalysts are known to the skilled worker.

To this end, for example, a normalized gene library of the above-described type is transferred into a suitable host organism, such as bacteria and here, for example, Escherichia coli, Salmonella spec., Streptomyces spec., Streptomyces nidulans, Streptomyces lividans, Bacillus subtilis, Lactococcus or Corynebacterium or yeasts such as, for example Pichia or Saccharomyces. The host organisms are listed here by way of example and not by way of limitation of the present invention. The transformed microorganisms obtained are then cultured on a nutrient medium (e.g. LB-agar plates) to which possible substrates of an enzyme class of interest, such as, for example, esterases, lipases, oxygenases etc., have been added, for selection of novel biocatalysts. Nutrient media which may be used are also selective media to which toxic substances, for example, have been added. By way of growth of the transformed microorganisms, formation of a lysis zone, turbidity of the culture medium, a color reaction or other conceivable reactions, it is then possible to select those transformants which contain with high probability a novel biocatalyst of microorganisms from soil samples, which were previously not culturable in the laboratory. The normalized DNA can then be (re)isolated from the selected transformants, sequenced and further characterized, resulting in the availability of appropriately novel genes coding for novel biocatalysts with an economically interesting application range.

It is also conceivable to identify the genes coding for novel biocatalysts via hybridization experiments of the normalized gene library with suitable DNA or RNA probes or antibodies.

The present invention is illustrated in more detail on the basis of the following examples which, however, are not limiting to the present invention:

### EXAMPLES

### 1. General Information

General genetic-engineering or molecular-genetic procedures such as, for example, restriction mixtures, clonings, growth and selection of transgenic organisms, agarose gel electrophoreses, preparation of primers, PCR, etc. were carried out using common methods according to Maniatis et al. (Maniatis V., Sambrook J, Fritsch E F & Maniatis V (1989). Molecular Cloning: A Laboratory Manual. Vol. I-III. Cold Spring Harbour Laboratory Press).

### 2. DNA Isolation

a) DNA isolation, modified according to Zhou et al. (1996, Appl. Environ. Microbiol., 62(2): 316-322), for cloning in E. coli

With the aid of this method, it is possible to isolate DNA from soil samples with high yield, but actinomycetes are hardly disrupted.

Buffer:

Extraction buffer: 100 mM Tris-HCl, pH 8

100 mM Na-EDTA, pH 8

100 mM sodium phosphate, pH 8

1.5 M NaCl

1% CTAB (hexadecylmethylammonium bromide)

1 g of soil sample is admixed with 2.6 ml of extraction buffer, vortexed and subjected to 3 “freeze-thaw” cycles (liquid nitrogen →+65° C.). 50 μl of proteinase K (20 mg/ml) are then added and the mixture is incubated with shaking at 37° C. for 30 min. This is followed by adding 300 μl of a 20% strength SDS solution and incubating at 65° C. for 2 hours. The mixture is then centrifuged at 5000 rpm for 10 min and the supernatant is collected. The pellet is washed once with 2 ml of extraction buffer and 250 μl of 20% SDS (incubation at 65° C. for 10 min, centrifugation at 5000 rpm for 10 min). The combined supernatants are admixed with 1/10 volume of 10% CTAB and centrifuged at 5000 rpm for 10 min. The aqueous phase is extracted with 1 volume of chloroform. The aqueous phase is then precipitated with 0.7 volume of isopropanol. The pellet is taken up in 100 μl of TE.

b) DNA isolation, modified according to Moré (1994, Appl. Environ. Microbiol., 60(5): 1572-1580) with direct purification

This method can be used to isolate DNA from all microorganisms, including actinomycetes, with high yield.

Buffer:

Sodium phosphate buffer: 100 mM, pH 8

10% SDS buffer: 100 mM NaCl

500 mM Tris-HCl, pH 8

10% (w/v) SDS

L6 buffer: 5 M guanidine thiocyanate

50 mM Tris-HCl, pH 8

25 mM NaCl

20 mM EDTA

1.3% Triton X-100

L2 buffer: 5 M guanidine thiocyanate

50 mM Tris-HCl, pH 8

25 mM NaCl

Silica: suspend 4.8 g of silica in 40 ml of H₂O, allow to settle for 24 h

remove 35 ml of supernatant, increase volume to 40 ml with H₂O, allow to settle for 30 min

remove supernatant, increase volume to 40 ml, allow to settle overnight

remove 36 ml, add 48 μl of 30% strength HCl to the “pellet”, vortex,

divide into aliquots, store in the dark at RT

0.5 g of soil sample is admixed with 0.5 ml of 100 mM sodium phosphate buffer, pH 8, 250 μl of SDS buffer and 2 g of glass beads (Ø 0 0.1 mm-0.25 mm) and mixed by vortexing. After shaking in a Retsch mill at a frequency of 1800 min⁻¹ and an amplitude of 80 for 10 min, the mixture is subsequently centrifuged at room temperature and 14 000 rpm for 10 min. The supernatant is removed, and the pellet is admixed with 300 μl of sodium phosphate buffer, pH 8 and incubated in an ultrasound bath for 2 min and then removed by centrifugation at 14 000 rpm for 3 min. The combined supernatants are admixed with ⅖ volume of 7.5 M ammonium acetate, vortexed and incubated on ice for 5 min. This is followed by centrifugation at 14 000 rpm for 3 min, and the supernatant (650 μl) is admixed with 1/10 volume of silica (65 μl) and 2 volumes of buffer L6 (1.3 ml), mixed and centrifuged at 3 000 rpm for 3 min. The supernatant is removed by decanting, has 1.3 ml of buffer L2 added to it, then is mixed thoroughly by way of shaking. The mixture is then centrifuged at 3 000 rpm for 3 min, the supernatant is discarded and the pellet is washed with 1.5 ml of 70% ethanol (shaking, centrifugation at 3 000 rpm for 3 min) and dried. The DNA is eluted by adding 100 ml of TE, incubating with shaking at 56° C. for 10 min and centrifuging at 14 000 rpm for 1 min, and the supernatant is then carefully removed and transferred to a fresh Eppendorf vessel.

### 3. DNA Purification

For this, the following materials are used:

Exclusion chromatography columns: CHROMA SPIN™-1000 Column, CLONTECH Laboratories, Inc.; elution buffer: 10 mM Tris/HCl pH 8.5.

Before applying the isolated soil DNA to the CHROMA SPIN™-1000 column, the latter is rinsed with elution buffer (10 mM Tris/HCl [pH 8.5]) according to the manufacturer's instructions.

Up to 100 μl of the isolated soil DNA are applied to the CHROMA SPIN-1000 column and eluted according to the manufacturer's instructions. The degree of purification can be estimated by recording UV/VIS spectra before and after the purification step. A decrease in absorption over the entire UV/VIS region indicates a reduction in the concentration of humic substances in the sample solution (the absorption band of nucleic acids is between approx. 230 and 300 nm).

### 4. DNA Quantification Using Fluorescent Dyes

The following materials were used:

TE buffer: 10 mM Tris/HCl [pH 7.5], 1 mM EDTA [pH 8.0]

SYBR Green I: Sigma, working solution: 1:3750 (diluted with TE)

calf thymus DNA: Sigma, stock solution and standards prepared in TE buffer stock solution: 100 μg/ml standards: 0.0 μg/ml; 0.3 μg/ml; 0.7 μg/ml, 1.0 μg/ml; 2.0 μg/ml, 3.0 μg/ml; 4.0 μg/ml; 5.0 μg/ml

fluorimeter: excitation wavelength: 485 nm; emission wavelength: 535 nm (optimal: 524 nm)

Preliminary Experiment:

The post-digestion crude extract is firstly diluted to such an extent (e.g. 1:50, ultimately depending on the dsDNA content in the soil sample and on the digestion method) so that, on the one hand, absorption at 535 nm and 485 nm is ≦0.05, but that, on the other hand, there is still enough dsDNA in the diluted sample so as to ensure accurate measurement. For this purpose, a preliminary experiment is carried out in which the fluorescence levels of different levels of dilution of the crude extract are determined. These fluorescence levels must, of course, be inside the calibration line used and be at least 5-6 times the fluorescence of the calibration line blank.

Addition of standard: (addition of DNA standards to aliquots of the diluted sample)

50 μl of diluted sample (see 1. )

+50 μl of DNA standard (0.0-5 μg/ml; calf thymus DNA)

+150 μl of SYBR Green I (1:3750 in TE, pH 7.5)

Calibration Line:

50 μl of TE

50 μl of DNA standard (0.3-5 μg/ml; calf thymus DNA)

+150 μl of SYBR Green I (1:3750 in TE, pH 7.5)

Doping of the crude extract with calf thymus DNA to determine the amount recovered:

An aliquot of the crude extract is doped with a DNA solution of a known concentration. Example: 300 μl of digested crude extract are admixed with 5 μl of calf thymus DNA (100 μg/ml). This doped aliquot is diluted in the same way as the sample under 2. and, furthermore, the dsDNA concentration is determined according to the standard addition method (see 2. (for “diluted sample”, now use “doped sample”)).

Reaction Conditions/Measurement Parameters:

Reaction time: 10 min (in the dark)

Temperature: room temperature

Excitation wavelength: 485 nm

Emission wavelength: 535 nm

Standard microtiter plates (ideally with black wells)

Evaluation (by Way of Example):

In the preliminary experiment, a dilution of the crude extract of 1:30 was determined as sufficient (Abs(535 nm)=0.008; (Abs(485 nm)=0.021);

calculation of the slope correction factor K:

a) the slope of the calibration line,

m(calib.),

is calculated from the information given above under “calibration line”;

b) the slope of the calibration line,

m(sample) or m(doped sample),

is calculated from the information given above under “addition of standard”;

c) slope correction factor K=m(calib.)/m(sample) (or m(doped sample));

d) the fluorescence levels F from the addition of standard of the sample or of the doped sample are multiplied with the slope correction factor K [symbol] F_{corr.}

e) the corrected fluorescence levels F_{corr.} are used to determine the dsDNA concentration in the sample or in the doped sample according to the usual evaluation method for standard addition methods.

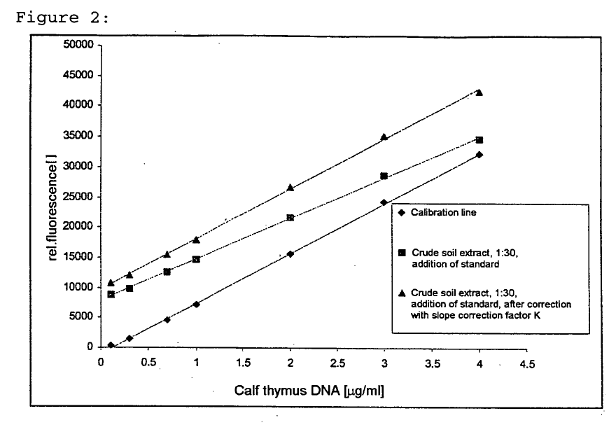

An example of the evaluation of a soil crude extract is depicted in Table 1 and FIG. 2.

### 5. DNA Fragmentation

The DNA was fragmented according to common laboratory practice. In detail, the genomic DNA is digested here with Hsp92II (Promega) with addition of 10 μg/μl nonacetylated BSA (DNAse-free, from Sigma) at 37° C. The reaction is stopped by adding 1/10 volume of EDTA (0.5 M, pH 8.0). The exact reaction times and amounts of enzyme and BSA required for limited digestion strongly depend on the DNA batches and must therefore be specifically determined in preliminary experiments. The appropriate procedures are familiar to the skilled worker. The digested DNA is precipitated with isopropanol, taken up in H₂O and fractionated via a 0.8% strength agarose gel. The size range of 3-5 kb is purified from the gel with the aid of QIAquick columns (Qiagen).

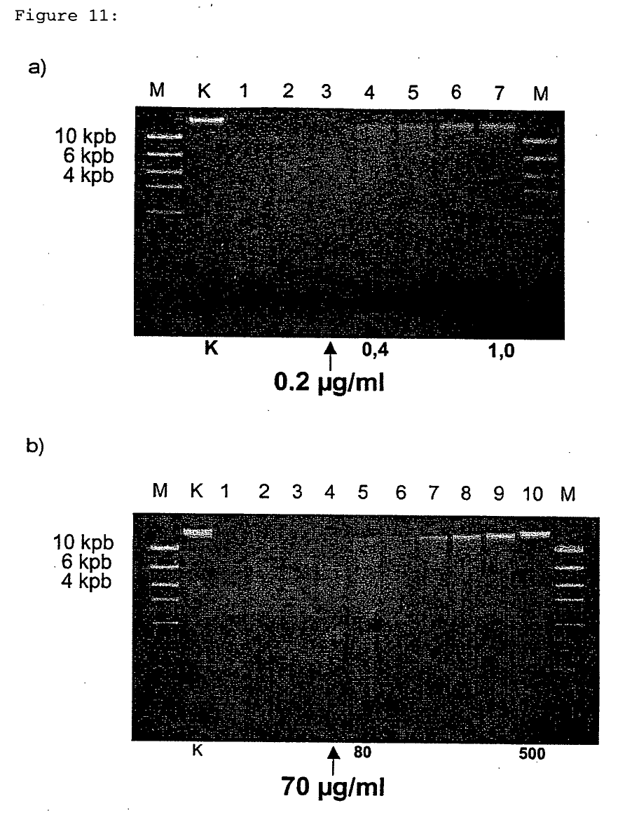

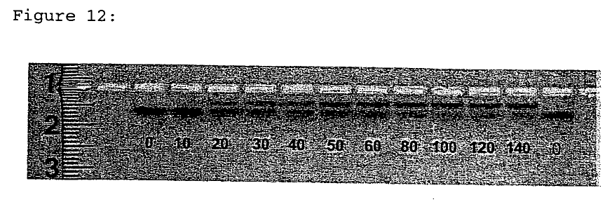

The positive effect of nonacetylated BSA with respect to restriction endonucleases was investigated by difference spectroscopic studies and with the aid of band shift experiments. It was shown that nonacetylated BSA interacts with humic acids (commercial humic acids (Fluka) were used) (FIG. 11). The addition of nonacetylated BSA to the reaction mixture enables genomic DNA to be digested in the presence of higher concentrations of humic substances, compared to carrying out the reaction without BSA addition. In the case of Sau 3AI restriction endonuclease, the concentration of humic acids may be approx. 350 times higher when nonacetylated BSA is added to the reaction mixture (minimum inhibiting concentration [MIC] of humic acid with no BSA addition: approx. 0.2 μg/ml, MIC of humic acid with BSA addition: approx. 70 μg/ml; determined by way of example for commercial humic acids (Fluka, lot 45729/1))(FIG. 12). However, the restriction endonucleases react with different sensitivity with respect to the humic substances and therefore also have different MICs. An MIC of humic substances of approx. 0.2 μg/ml with no BSA addition was found for the enzyme Hsp 92II. The addition of nonacetylated BSA increased the MIC to approx. 3.0 μg/ml humic substances (factor: 15). It is also necessary to determine the optimal BSA concentration for each restriction enzyme. Said concentration is for Sau 3AI 8 μg/μl of reaction solution (final), and for Hsp 92II an optimal BSA concentration of 2 μg/μl of reaction mixture was found. A further increase in BSA concentration had no positive effect on the MIC.

In order to save optimization steps, a final BSA concentration of 10 μg/μl is generally recommended. After the addition of nonacetylated BSA to the reaction mixture (enzyme not added yet), a preincubation time of 5 min should be observed. This step is intended to ensure that the nonacetylated BSA has sufficient time to react completely with the humic substances.

### 6. Ligation with Linkers Suitable for Cloning and Amplification

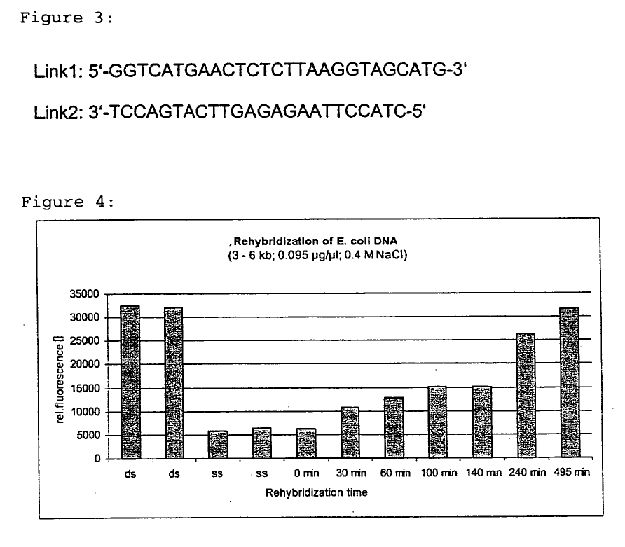

FIG. 3 depicts suitable linkers according to SEQ ID No. 1 and 2. The linkers are ligated with the fragmented DNA from soil bacteria according to the manufacturer's instructions (LigaFast Rapid DNA Ligation System from Promega).

### 7. DNA Normalization

For this, the following materials were used:

TE buffer: 10 mM Tris/HCl [pH 7.5], 1 mM EDTA [pH 8.0]

SYBR Green I: Sigma, working solution: diluted 1:4 000 with TE buffer

3 M NaCl solution

urea solutions: 1 M; 2 M

fragmented DNA (3-10 kbp, partial digest): 0.1 μg/μl (in the reaction vessel)

200 μl reaction vessels

preheat pipette tips to 65° C.

fluorimeter: excitation wavelength: 485 nm

emission wavelength: 535 nm

(optimal:524 nm)

thermocycler with heatable lid

The sample to be normalized (volume: 30 μl; 0.1 μg/μl of DNA (3-10 kbp), 0.4 M NaCl) is first heated to 65° C. Thereafter, an aliquot of 2 μl was removed and transferred into 18 μl of 1 M urea solution. This solution is immediately stored on ice (=N_{dsDNA}). The sample is denatured at 95° C. for 5 min. After cooling of the sample to rehybridization temperature (65° C.), another aliquot of 2 μl is removed, transferred into 18 μl of 1 M urea solution (=N₀) and immediately stored on ice. During the entire rehybridization period, further aliquots are removed at different times. The fluorescence measurement is carried out as described below:

20 μl of sample (N_{dsDNA}, N₀, N₁ₕ, . . . )

+100 μl of 2 M urea solution

+80 μl of SYBR Green I (1:4 000 in TE) are introduced into standard microtiter plates (ideally with black wells) and incubated in the dark at room temperature for 10 min; excitation wavelength: 485 nm; emission wavelength: 535 nm. The evaluation is carried out by plotting the relative fluorescence as a function of the renaturation time. FIGS. 4, 5 and 6 depict the result in the form of a bar chart.

### 8. ssDNA Fractionation Via Hydroxyapatite

a) Removal of ssDNA at room temperature in the batch process

Chemicals and Apparatus

Hydroxyapatite: Bio-Gel HTP hydroxyapatite or DNA grade Bio-Gel HTP hydroxyapatite (Biorad)

ssDNA elution buffer: 0.17 M NaPO₄ [pH 6.8]

dsDNA elution buffer: 0.34 M NaPO₄ [pH 6.8]

Procedure

After normalization, the DNA solution is left cooling at room temperature for 5 min. If pH and phosphate concentration of the rehybridization buffer do not correspond to the conditions of the ssDNA elution buffer, the DNA solution is adjusted to ssDNA-elution buffer conditions (0.17 M NaPO₄ [pH 6.8]) by adding higher-concentrated NaPO₄ buffer. For binding to the hydroxyapatite, 50 μl of a hydroxyapatite suspension (in ssDNA elution buffer), for example, are added to the DNA solution, the mixture is mixed briefly (Vortex), incubated at RT for 1 min, mixed again and incubated at RT for 1 min. After subsequent centrifugation (2-5 s, RT), the supernatant which contains the majority of ssDNA is removed.

The remaining ssDNA is eluted by adding 30 μl of ssDNA elution buffer, mixing, centrifuging for 2-5 s and removing the supernatant. This procedure is repeated at least 5 times.

b) Removal of ssDNA at room temperature using hydroxyapatite as spin column

Chemicals and Apparatus

As under 8a)

empty Mobicol columns with small filters (diameter: 2.7 mm, pore size: 35 μM) from MoBiTec (Göttingen).

Procedure

Denaturation, renaturation, cooling to RT and adjustment to ssDNA-elution buffer conditions are carried out as stated under (8a).

The hydroxyapatite spin column is prepared by pipetting 50 μl of a hydroxyapatite suspension (in ssDNA elution buffer) into the Mobicol column and centrifuging briefly. The DNA solution is applied to the hydroxyapatite spin column, and DNA and hydroxyapatite are carefully mixed. After brief centrifugation at RT, the eluate contains the majority of ssDNA.

The remaining ssDNA may be recovered by elution with 150 μl of ssDNA elution buffer.

### 9. ssDNA Amplification for Cloning Into Suitable Gene Constructs or Vectors

ssDNA is amplified using the Expand long template PCR system from Roche according to the manufacturer's instructions. The primer used is the oligonucleotide Link1 which is also used in the linker. Unspecific PCR products and the primers which are not required during PCR are removed via an agarose gel (0.8% agarose). The size range of 3-5 kb is eluted with the aid of QIAquick columns (Qiagen). The PCR fragments are digested with I-Ppol and purified via QIAquick columns (Qiagen). The eluate is used for ligation with the appropriately pretreated gene construct PSCR which has been linearized with I-Ppol (Promega) and dephosphorylated with CIAP (Promega). The construct is then used to transform E. coli BL21(DE3)pLysS.

### 10. Preparation of the pSCR Gene Construct

The pSCR plasmid was prepared by digesting the pBR322 plasmid with HindIII (NEB) and Mva12691 (Fermentas) and isolating a 3033 bp fragment.

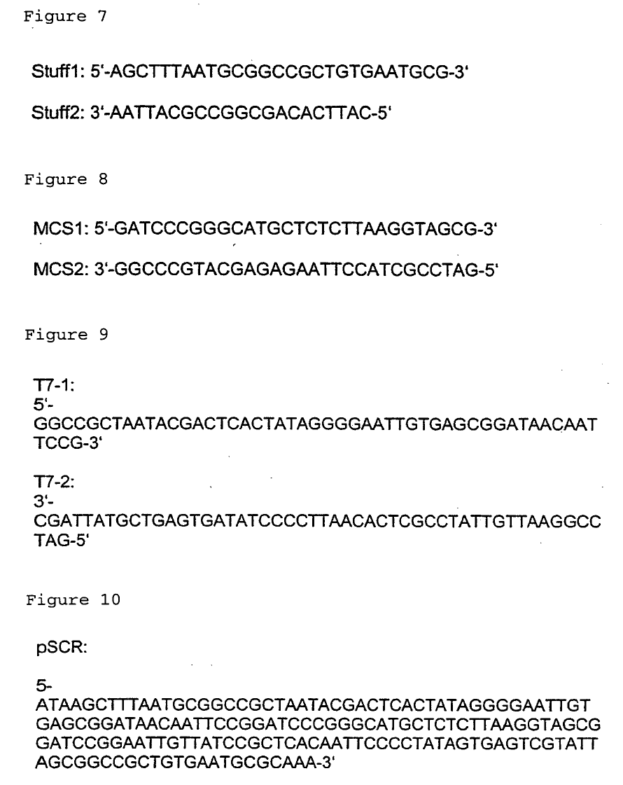

First, the stuffer stuff1 or stuff2 according to SEQ ID No. 3 and 4, respectively, was ligated into this vector. The resulting plasmid was cleaved with NotI and the promoter regions T71 and T72 (from plasmid pET-15b, Promega) were ligated, resulting in two T7 promoters in opposite orientation to one another. The oligonucleotides for the multiple cloning sites MCS1 and MCS2 according to SEQ ID No. 5 and 6, respectively, which have an I-Ppol cleavage site, were then ligated into the BamHI cleavage site located between said promoters. The corresponding sequence sections and the total sequence of the pSCR gene construct are depicted in FIGS. 7-9 and in FIG. 10, respectively.

### 11. Screening of the Normalized Gene Library for Identifying Genes Coding for Novel Biocatalysts from Soil Samples

After transformation, the screening, for example for esterases, is carried out by plating the freshly transformed cells directly on (turbid) tributyrin plates (1.5% agar, 1% (w/v) tributyrin, LB medium; homogenized prior to autoclaving). After incubation at 37° C. for 24 hours, the turbid plates are stored at 4° C. and checked each day for the formation of a clear (lysis) zone. Clones around which a lysis zone has formed are identified as potentially esterase-positive. These clones are transferred from the selective medium plate to complete medium, cloned and subjected to further analyses.

Table and Figure Legends:

Table 1: Example of the evaluation of fluorimetric quantification of dsDNA from digested soil samples without prior purification.

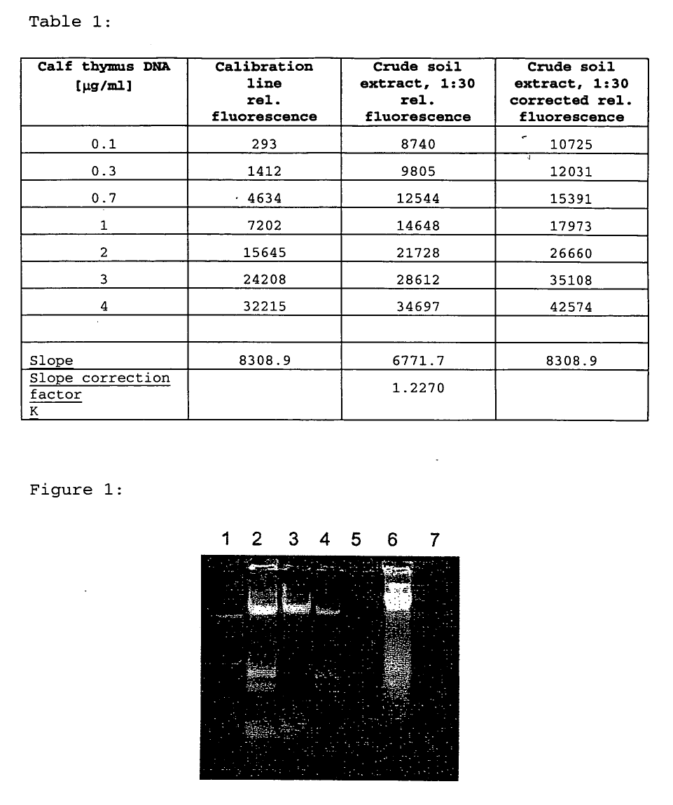

FIG. 1: Gel electrophoretic fractionation of DNA isolated from soil bacteria. Lane 1: size markers (kb), lane 2: Arthrobacter—Moré, lane 3: Pseudomonas—Moré, lane 4: Rhodococcus—Moré, lane 5: Arthrobacter—Zhou, lane 6: Pseudomonas—Zhou, lane 7: Rhodococcus—Zhou.

FIG. 2: Graphical representation of the correction method for fluorimetric quantification of dsDNA in digested soil samples.

FIG. 3: Nucleotide sequences according to SEQ ID No. 1 and 2 corresponding to the preferably used linkers link1 and link2 for preparing a preferred gene construct.

FIG. 4: Representation of the rehybridization of E. coli DNA by way of plotting the relative fluorescence as a function of the rehybridization time.

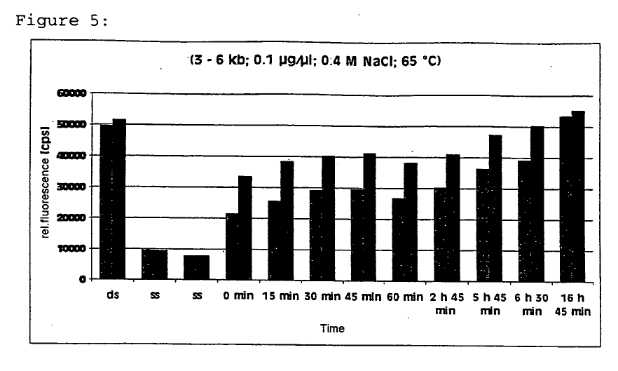

FIG. 5: Representation of the rehybridization of Pseudomonas DNA and of a mixture of Pseudomonas and E. coli DNA in a 2:1 ratio by way of plotting the relative fluorescence as a function of the rehybridization time.

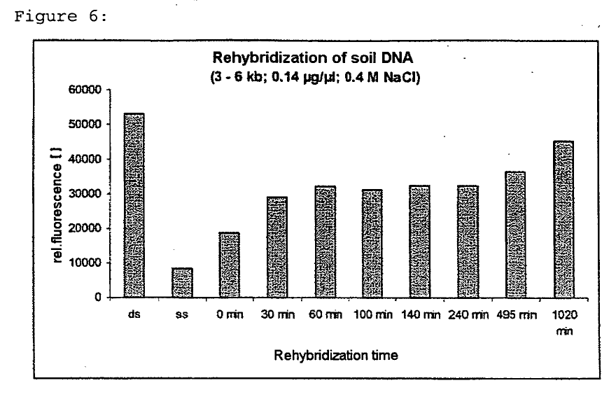

FIG. 6: Representation of the rehybridization of soil sample DNA by way of plotting relative fluorescence as a function of rehybridization time.

FIG. 7: Nucleotide sequences according to SEQ ID No. 3 and 4, corresponding to the preferably used stuffers of stuff1 and stuff2 for preparing a preferred gene construct.

FIG. 8: Nucleotide sequences according to SEQ ID No. 5 and 6, corresponding to the preferably used multiple cloning sites MCS1 and MCS2 for preparing a preferred gene construct.

FIG. 9: Nucleotide sequences according to SEQ ID No. 7 and 8, corresponding to the preferably used T7 promoters T7-1 and T7-2 for preparing a preferred gene construct.

FIG. 10: Nucleotide sequence according to SEQ ID No. 9 of a preferred gene structure pSCR comprising stuffer sequences stuff1 and stuff2, two opposite promoter sequences of the T7 promoter from pET-15b plasmid from Promega, which is regulated by the lac operator, and also multiple cloning sites comprising at least the rarely occurring recognition sequence for the Physarum polycephalum restriction endonuclease I-Ppol.

FIG. 11: Minimum inhibiting concentration (MIC) of humic acids (Fluka) for the Sau3AI restriction enzyme without (a) and with (b) addition of nonacetylated BSA. 1 μg of genomic E. coli DNA was digested with Sau3AI (0.3 μg, absolute) in the presence of increasing concentrations of humic acids.

a) Lane M: size marker; lane K: without Sau3AI; lanes 1-7: 0; 0.1; 0.2; 0.4; 0.6; 0.8; 1.0 μg/ml humic acids.

b) Lane M: size marker; lane K: without Sau3AI; lanes 1-10: 0; 50; 60; 70; 80; 90; 100; 150; 200; 500 μg/ml humic acids.

Without the presence of nonacetylated BSA (a), the DNA was still digested in the presence of 0.2 μg/ml humic acids. At higher humic acid concentrations, the enzyme was very strongly inhibited. With addition of 10 μg/μl (final conc.) nonacetylated BSA to the reaction mixture, the genomic DNA was still completely digested in the presence of 70 μg/ml humic acid.

FIG. 12: Bandshift assay for detecting the interaction of humic acids (Fluka) and nonacetylated BSA. 20 μg of humic acids were incubated with increasing nonacetylated BSA contents and electrophoretically analyzed (1.0% strength agarose gel). In the presence of nonacetylated BSA, an additional band appears which is not detectable in the control band (0 μg of BSA).

1. A method for preparing a normalized gene library from nucleic acid extracts of soil samples, which method comprises
a) extracting nucleic acids from living organisms present in soil samples;
b) fragmenting said nucleic acids;
c) quantifying the nucleic acid fragments by means of fluorescent dyes;
d) normalizing said nucleic acid fragments, first denaturing the latter and then monitoring the course of renaturation by means of fluorescent dyes;
e) separating, after renaturation has ended, the double-stranded nucleic acids from the single-stranded nucleic acids by adsorption chromatography, the amount of nucleic acid species present in the fraction of the single-stranded nucleic acids being frequently approximately equal (normalized); and
f) generating the gene library by cloning the normalized nucleic acid species into a vector.

2. A method as claimed in claim 1, wherein nucleic acids are extracted from soil-dwelling organisms which cannot be cultured in the laboratory.

3. A method as claimed in either of claims 1 or 2, wherein nucleic acids are selectively isolated from actinomycetes.

4. A method as claimed in claim 1, wherein nonacetylated bovine serum albumin at concentrations of about 1-15 μg, preferably of about 2-12 μg, and particularly preferably of about 10 μg, per μl of restriction mixture is used in fragmentation of the nucleic acids.

5. A method as claimed in claim 1, wherein the fragmented nucleic acids are linked to linkers which have at least one recognition site for a rarely occurring restriction endonuclease.

6. A method as claimed in claim 5, wherein the linkers which have a recognition site for the restriction enzyme I-Ppol are used.

7. A method as claimed in claim 6, wherein step f) employs a vector which has at least one recognition site for a rarely occurring restriction endonuclease which is compatible with the recognition site in the linkers.

8. A method as claimed in claim 1, wherein the nucleic acids extracted from soil samples and/or their fragments are quantified in step c) using fluorescent dyes, preferably SYBR-Green-I.

9. A method as claimed in claim 1, wherein the time course of renaturation of the previously denatured nucleic acid fragments in step d) is fluormetrically monitored using DNA-specific fluorescent dyes, preferably SYBR-Green-I.

10. A method as claimed in claim 1, wherein adsorption chromatography in step e) is carried out by means of hydroxyapatite.

11. A method as claimed in claim 1, wherein the adsorption chromatography in step e) is carried out in a batch process.

12. A method as claimed in claim 1, wherein single stranded nucleic acids and double stranded nucleic acids are fractionated at from 20 to 60° C., preferably at from 20 to 30° C., and particularly preferably at 22° C.

13. A method as claimed in claim 1, wherein single stranded nucleic acids and double stranded nucleic acids are fractionated in an NaPCU buffer having a concentration of 0.15-0.17 M.

14. A method as claimed in claim 1, wherein the adsorption chromatography in step e) is carried out in spin columns.

15. A gene structure, comprising at least one multiple cloning site with at least one rarely occurring recognition site for restriction endonucleases, a primer-binder site-and/or a T7-polymerase recognition site whose activity is regulated via the lac operator.

16. A gene structure as claimed in claim 15, comprising at least one recognition site for the restriction enzyme I-Ppol.

17. A gene structure as claimed in claim 16, which has a sequence according to SEQ ID No. 10.

18. A vector, comprising at least one gene structure as claimed in any one of claims 15 to 17 and also additional nucleotide sequences for selection, for replication in the host cell or for integration into the host cell genome.

19. The use of the rarely occurring recognition site for the I-Ppol restriction endonuclease for preparing a gene structure as claimed in either of claims 15 or 16.

20. The use of the normalized gene library prepared by a method as claimed in claim 1 for the selection of genes coding for biocatalysts of soil-dwelling microorganisms.

21. The use of the rarely occurring recognition site for the I-Ppol restriction endonuclease for preparing a vector as claimed in claim 18.


SEQUENCE LISTING (9 sequences)

SEQ ID NO: 1 - DNA, 27 bp, Artificial Sequence
  linker, 5′-3′
ggtcatgaactctcttaaggtagcatg

SEQ ID NO: 2 - DNA, 24 bp, Artificial Sequence
  linker, 3′-5′
tccagtacttgagagaattccatc

SEQ ID NO: 3 - DNA, 27 bp, Artificial Sequence
  stuffer, 5′-3′
agctttaatgcggccgctgtgaatgcg

SEQ ID NO: 4 - DNA, 21 bp, Artificial Sequence
  stuffer, 3′-5′
aattacgccggcgacacttac

SEQ ID NO: 5 - DNA, 29 bp, Artificial Sequence
  multiple cloning site, 5′-3′
gatcccgggcatgctctcttaaggtagcg

SEQ ID NO: 6 - DNA, 29 bp, Artificial Sequence
  multiple cloning site, 3′-5′
ggcccgtacgagagaattccatcgcctag

SEQ ID NO: 7 - DNA, 51 bp, Artificial Sequence
  T7-Promotor, 5′-3′
ggccgctaatacgactcactataggggaattgtgagcggataacaattccg

SEQ ID NO: 8 - DNA, 51 bp, Artificial Sequence
  T7-Promotor, 5′-3′
cgattatgctgagtgatatccccttaacactcgcctattgttaaggcctag

SEQ ID NO: 9 - DNA, 165 bp, Artificial Sequence
  pSCR gene construct“
ataagctttaatgcggccgctaatacgactcactataggggaattgtgagcggataacaattccggatcccgggcatgctctcttaaggtagcggatccggaattgttatccgctcacaattcccctatagtgagtcgtattagcggccgctgtgaatgcgcaaa